(19) United States
(12) Patent Application Publication
(10) Pub. No.: US 2025/0228866 A1
(43) Pub. Date: Jul. 17, 2025

(54) CRYSTALLINE FORM OF (R)-1-(3-(3-(4-AMINOPYRIMIDIN-2-YL)-5-CHLOROPHENYL)MORPHOLINO)PROP-2-EN-1-ONE

(71) Applicant: Vividion Therapeutics, Inc., San Diego, CA (US)

(72) Inventor: Wei LIN, San Diego, CA (US)

(21) Appl. No.: 19/096,630

(22) Filed: Mar. 31, 2025

Related U.S. Application Data
(60) Provisional application No. 63/658,055, filed on Jun. 10, 2024.

(51) Int. Cl. A61K 31/5377; C07D 413/10

(52) U.S. Cl. CPC A61K 31/5377; C07D 413/10

(57) ABSTRACT

The present disclosure relates to a solid crystalline form of (R)-1-(3-(3-(4-aminopyrimidin-2-yl)-5-chlorophenyl)morpholino)prop-2-en-1-one and to methods for preparing such crystalline form. (R)-1-(3-(3-(4-aminopyrimidin-2-yl)-5-chlorophenyl)morpholino)prop-2-en-1-one is a potent KEAP1 modulator.

### CROSS-REFERENCE TO RELATED APPLICATION

The application claims the benefit of provisional application No. 63/658,055 filed Jun. 10, 2024, which is incorporated in its entirety herein.

### FIELD OF THE INVENTION

The present disclosure relates to a solid crystalline form of (R)-1-(3-(3-(4-aminopyrimidin-2-yl)-5-chlorophenyl)morpholino)prop-2-en-1-one and to a method for preparing such crystalline form.

### BACKGROUND OF THE INVENTION

(R)-1-(3-(3-(4-aminopyrimidin-2-yl)-5-chlorophenyl)morpholino)prop-2-en-1-one and the manufacture thereof has been disclosed in WO 2024/073587 to Weinstein et al. and US App. Pub. 2024/0226114 A1 to Weinstein et al., which are incorporated by reference herein in their entireties. (R)-1-(3-(3-(4-aminopyrimidin-2-yl)-5-chlorophenyl)morpholino)prop-2-en-1-one is a potent KEAP1 modulator and may be useful in the treatment of neurodegenerative diseases or cancer. (R)-1-(3-(3-(4-aminopyrimidin-2-yl)-5-chlorophenyl)morpholino)prop-2-en-1-one is represented by the structure:

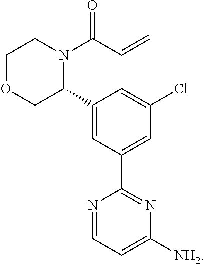 [SMILES: C=CC(=O)N1CCOC[C@H]1c1cc(Cl)cc(-c2nccc(N)n2)c1]

### SUMMARY OF THE DISCLOSURE

It has now been found that (R)-1-(3-(3-(4-aminopyrimidin-2-yl)-5-chlorophenyl)morpholino)prop-2-en-1-one can be obtained in a stable and substantially pure crystalline form by crystallization under certain conditions.

Thus, the present disclosure provides, as one aspect, (R)-1-(3-(3-(4-aminopyrimidin-2-yl)-5-chlorophenyl)morpholino)prop-2-en-1-one in crystalline form I.

In another aspect, the present disclosure provides (R)-1-(3-(3-(4-aminopyrimidin-2-yl)-5-chlorophenyl)morpholino)prop-2-en-1-one in crystalline form I as defined herein, substantially free of any other crystalline or amorphous form of (R)-1-(3-(3-(4-aminopyrimidin-2-yl)-5-chlorophenyl)morpholino)prop-2-en-1-one.

The crystalline form I is stable during pharmaceutical processing and storage and is therefore particularly suitable for the preparation of pharmaceutical formulations.

### DESCRIPTION

### Brief Description of the Drawings

The patent or application file contains at least one drawing executed in color. Copies of this patent or patent application publication with color drawing(s) will be provided by the Office upon request and payment of the necessary fee.

FIG. 1 shows the X-ray powder diffraction pattern of the crystalline form I of (R)-1-(3-(3-(4-aminopyrimidin-2-yl)-5-chlorophenyl)morpholino)prop-2-en-1-one obtained in Example 1.

FIG. 1a shows the computer-simulated X-ray powder diffraction (XRPD) pattern based on single crystal data of (R)-1-(3-(3-(4-aminopyrimidin-2-yl)-5-chlorophenyl)morpholino)prop-2-en-1-on obtained in Example 2.

FIG. 2 shows the DSC thermogram of the crystalline form I (R)-1-(3-(3-(4-aminopyrimidin-2-yl)-5-chlorophenyl)morpholino)prop-2-en-1-one obtained in Example 1.

FIG. 2a shows the TGA thermogram of the crystalline form I (R)-1-(3-(3-(4-aminopyrimidin-2-yl)-5-chlorophenyl)morpholino)prop-2-en-1-one obtained in Example 1.

FIG. 3 show the packing diagram of (R)-1-(3-(3-(4-aminopyrimidin-2-yl)-5-chlorophenyl)morpholino)prop-2-en-1-one molecules in a single crystal of crystalline form I (a axis) obtained in Example 2.

FIG. 4 show the packing diagram of (R)-1-(3-(3-(4-aminopyrimidin-2-yl)-5-chlorophenyl)morpholino)prop-2-en-1-one molecules in a single crystal of crystalline form I (b axis) obtained in Example 2.

FIG. 5 show the packing diagram of (R)-1-(3-(3-(4-aminopyrimidin-2-yl)-5-chlorophenyl)morpholino)prop-2-en-1-one molecules in a single crystal of crystalline form I (c axis) obtained in Example 2.

### DETAILED DESCRIPTION OF THE INVENTION

Crystalline form I of (R)-1-(3-(3-(4-aminopyrimidin-2-yl)-5-chlorophenyl)morpholino)prop-2-en-1-one has been characterized by X-ray powder diffraction (XRPD) studies.

Accordingly, in one aspect, the present disclosure provides crystalline form I of (R)-1-(3-(3-(4-aminopyrimidin-2-yl)-5-chlorophenyl)morpholino)prop-2-en-1-one having an X-ray powder diffraction pattern comprising characteristic peaks at about 16.7, 18.9, 20.5, 22.1, 25.4 degrees 2-theta.

In another aspect, the present disclosure provides crystalline form I of (R)-1-(3-(3-(4-aminopyrimidin-2-yl)-5-chlorophenyl)morpholino)prop-2-en-1-one having an X-ray powder diffraction pattern comprising characteristic peaks at about 9.6, 14.6, 16.7, 17.8, 18.9, 20.5, 22.1, 22.2, 25.4, 28.9 degrees 2-theta.

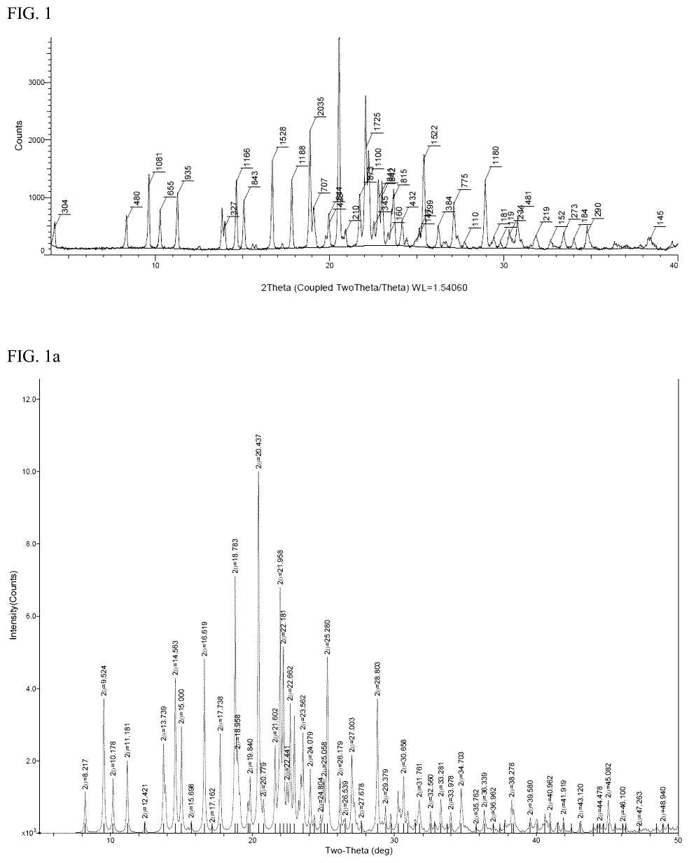

In still another aspect, the present disclosure provides crystalline form I of (R)-1-(3-(3-(4-aminopyrimidin-2-yl)-5-chlorophenyl)morpholino)prop-2-en-1-one characterized in that it provides an X-ray powder diffraction pattern substantially as illustrated in FIG. 1.

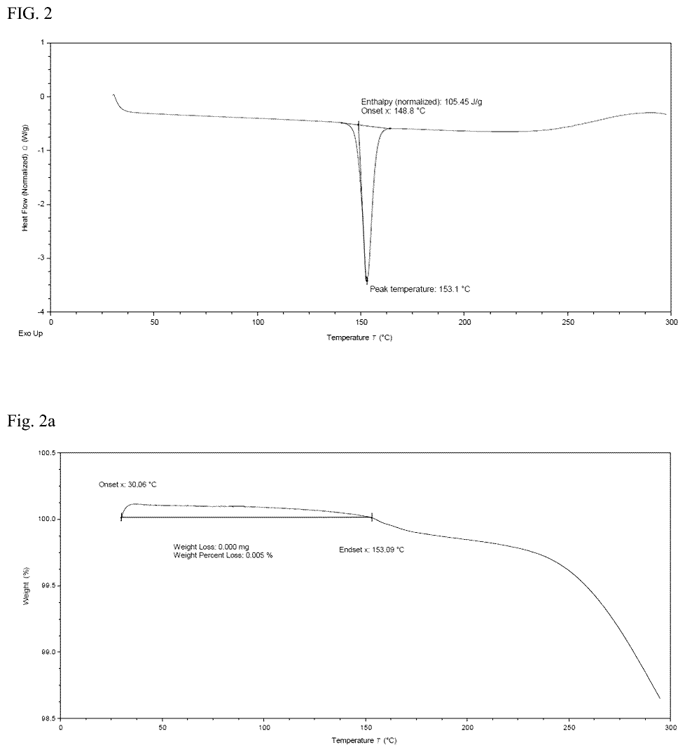

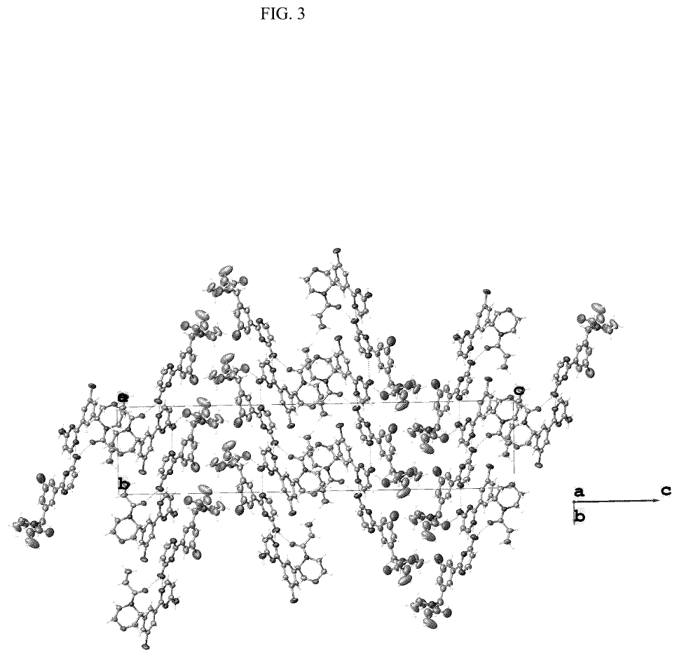

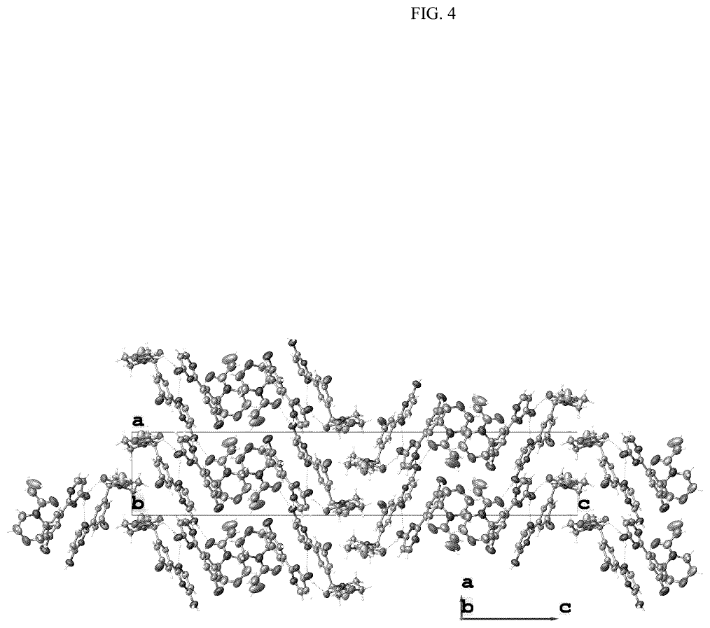

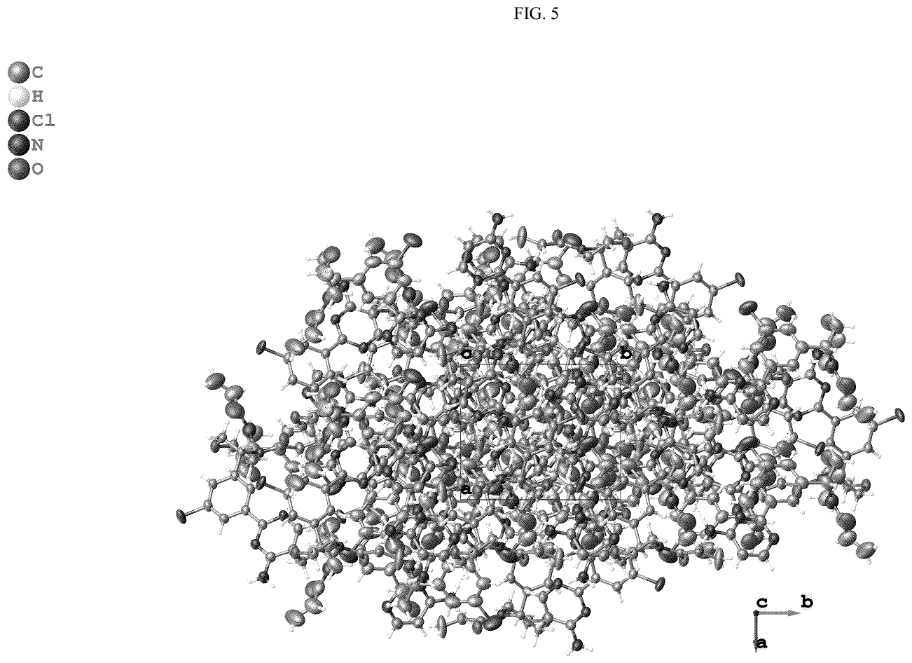

In yet another aspect, the present disclosure provides crystalline form I of (R)-1-(3-(3-(4-aminopyrimidin-2-yl)-5-chlorophenyl)morpholino)prop-2-en-1-one characterized in that it provides a DSC thermogram as illustrated in FIG. 2.

In still another aspect, the present disclosure provides crystalline form I of (R)-1-(3-(3-(4-aminopyrimidin-2-yl)-5-chlorophenyl)morpholino)prop-2-en-1-one characterized in that it has a melting point onset ranging from about 140 to 149° C.

In still another aspect, the present disclosure provides crystalline form I of (R)-1-(3-(3-(4-aminopyrimidin-2-yl)-5-chlorophenyl)morpholino)prop-2-en-1-one characterized in that it has a melting enthalpy of about 73 to 106 J/g.

It is recognized by the skilled person that the X-ray powder diffraction pattern peak positions referred to herein can be subject to variations of ±0.15 degrees 2-theta according to various factors such as temperature, concentration, and instrumentation used. Therefore, signals and peak positions are referred to herein as being at “about” specific values. Melting point onset and melting enthalpy are also dependent on various factors like purity, heating rate and instrumentation used. Therefore, data are referred to herein as being at “about” specific values, too.

TABLE 1
Melting point and enthalpy values of representative
batches prepared by example 1 and example 2.

| Batch | Method of Preparation | Melting Point (° C.) | Enthalpy (J/g) |
|---|---|---|---|
| A | Example 1 | 148.2 | 100.1 |
| B | Example 1 | 148.7 | 101.8 |
| C | Example 1 | 148.8 | 105.5 |
| D | Example 2a | 140.2 | 72.7 |

According to still another aspect, the present disclosure provides a process for preparing crystalline form I of (R)-1-(3-(3-(4-aminopyrimidin-2-yl)-5-chlorophenyl)morpholino)prop-2-en-1-one, comprising:
a) preparing a solution of (R)-1-(3-(3-(4-aminopyrimidin-2-yl)-5-chlorophenyl)morpholino)prop-2-en-1-one in a mixture of approximately 4 volumes (V) acetone and approximately 1 V water at approximately 50° C.;
b) adding approximately 6 V water over approximately 2 hours;
c) adding approximately 1 wt % seed crystals (obtained according to Example 2);
d) aging the reactor content for approximately 5 hours;
e) adding approximately 8 V water over approximately 11 hours;
f) stirring the resulting mixture for approximately 2 hours;
g) adjusting the mixture to approximately 0° C. over approximately 5 hours;
h) stirring the mixture for approximately 10 hours at 0° C.;
i) filtering;
j) rinsing the wet cake with approximately 3.8 V cold acetone/water mixture (approximately 1/3 v/v);
k) rinsing the wet cake with approximately 4 V water; and
l) drying at approximately 50 under vacuum for 1 approximately 15 hours.

The crystalline forms I of (R)-1-(3-(3-(4-aminopyrimidin-2-yl)-5-chlorophenyl)morpholino)prop-2-en-1-one is useful as medicament and can be formulated into pharmaceutical dosage forms, such as tablets for oral administration, by mixing with pharmaceutical excipients known in the art.

The disclosure is further illustrated by the following examples.

### EXAMPLE 1

### Crystallization of (R)-1-(3-(3-(4-aminopyrimidin-2-yl)-5-chlorophenyl)morpholino)prop-2-en-1-one

Water (approx. 6 V) is added over 2 hours to a solution of (R)-1-(3-(3-(4-aminopyrimidin-2-yl)-5-chlorophenyl)morpholino)prop-2-en-1-one in a mixture of acetone (approx. 4 V) and water (approx. 1 V) at approx. 50° C. Seed crystals obtained according to example 2 (approx. 1 wt %) is charged, the reactor contents are aged for approx. 5 hrs, then water (approx. 8 V) is charged over approx. 11 hrs.

The resulting mixture is stirred for approx. 2 hrs then adjusted to approx. 0° C. over approx. 5 hrs. The reactor contents are stirred for approx. 10 hrs at 0° C. then filtered. The wet cake is rinsed with cold acetone/water (approx. 1/3 v/v, 3.8 V), water (4 V), then dried at approx. 50° C. under vacuum for approx. 15 hrs.

### EXAMPLE 2

### Preparing a Single Crystal of (R)-1-(3-(3-(4-aminopyrimidin-2-yl)-5-chlorophenyl)morpholino)prop-2-en-1-one Big Enough for a X-Ray Structure Analysis

A 2 mg solution of (R)-1-(3-(3-(4-aminopyrimidin-2-yl)-5-chlorophenyl)morpholino)prop-2-en-1-one in 200 mL tetrahydrofuran/water (4:1) is kept in a 1 mL vial. The solution evaporates slowly at room temperature. Crystals of polymorphic form I were observed on the second day.

### EXAMPLE 2a

### Crystallization of (R)-1-(3-(3-(4-aminopyrimidin-2-yl)-5-chlorophenyl)morpholino)prop-2-en-1-one

R)-1-(3-(3-(4-aminopyrimidin-2-yl)-5-chlorophenyl)morpholino)prop-2-en-1-one was stirred in about 10 volumes of water at about 70° C. for about 16 hours. The mixture was cooled to about 15° C., filtered and dried to give Batch D.

### EXAMPLE 3

### X-Ray Diffraction Study of Crystalline Form of (R)-1-(3-(3-(4-aminopyrimidin-2-yl)-5-chlorophenyl)morpholino)prop-2-en-1-one

The crystalline form of (R)-1-(3-(3-(4-aminopyrimidin-2-yl)-5-chlorophenyl)morpholino)prop-2-en-1-one obtained in Example 1 was analyzed by X-ray powder (XRPD) diffraction method. The XRPD measurements were recorded on a Bruker D8 Advance diffractometer using a LYNXEYE detector with monochromatized CuKα1-radiation, a position sensitive detector, at generator settings of 40 kV and 40 mA. The samples were collected in continuous PSD mode. The scanning range was between 4° and 40° 2 theta with a 0.02° step at 0.12 sec/step.

TABLE 2
Peak list (Example 3)

| | Pos. | |
|---|---|---|
| Peak number | [°2 Θ] | Rel. Int. [%] |
| 1 | 4.181 | 8.40% |
| 2 | 8.317 | 13.30% |
| 3 | 9.608 | 30.00% |
| 4 | 10.26 | 18.20% |
| 5 | 11.257 | 25.90% |
| 6 | 13.89 | 9.10% |
| 7 | 14.64 | 32.30% |
| 8 | 15.082 | 23.40% |
| 9 | 16.702 | 42.30% |
| 10 | 17.828 | 32.90% |
| 11 | 18.874 | 56.40% |
| 12 | 19.088 | 19.60% |
| 13 | 19.987 | 11.70% |
| 14 | 19.935 | 15.10% |
| 15 | 20.531 | 100.00% |
| 16 | 20.869 | 5.80% |
| 17 | 21.735 | 24.20% |
| 18 | 22.116 | 47.80% |
| 19 | 22.175 | 30.50% |
| 20 | 22.566 | 9.50% |
| 21 | 22.848 | 23.30% |
| 22 | 22.979 | 23.30% |
| 23 | 23.382 | 4.40% |
| 24 | 23.643 | 22.60% |
| 25 | 24.151 | 12.00% |
| 26 | 25.055 | 4.10% |
| 27 | 25.156 | 8.30% |
| 28 | 25.402 | 42.20% |
| 29 | 26.239 | 10.60% |
| 30 | 27.139 | 21.50% |
| 31 | 27.75 | 3.10% |
| 32 | 28.935 | 32.70% |
| 33 | 29.404 | 5.00% |
| 34 | 29.799 | 3.30% |
| 35 | 30.342 | 6.50% |
| 36 | 30.791 | 13.30% |
| 37 | 31.837 | 6.10% |
| 38 | 32.7 | 4.20% |
| 39 | 33.438 | 7.60% |
| 40 | 34.026 | 5.10% |
| 41 | 34.805 | 8.00% |
| 42 | 38.367 | 4.00% |

### EXAMPLE 4

### DSC Analysis of Crystalline Form of (R)-1-(3-(3-(4-aminopyrimidin-2-yl)-5-chlorophenyl)morpholino)prop-2-en-1-one

DSC thermograms were collected on a TA DSC250 using a few milligrams of material in a Tzero aluminum pan with a Tzero aluminum lid. Samples were scanned at 10° C. per minute under 50 mL per minute of nitrogen flow. The sample has a melting point onset of about 149° C. and enthalpy of about 105 J/g.

### EXAMPLE 5

### TGA Analysis of Crystalline Form of (R)-1-(3-(3-(4-aminopyrimidin-2-yl)-5-chlorophenyl)morpholino)prop-2-en-1-one

TGA thermograms were collected on a TA550. A few milligrams of material were loaded onto a pre-tared aluminum pan and heated at 10° C. per minute from 30° C. to 300° C.

### EXAMPLE 6

### Modeling an XRPD Spectrum of Crystals Obtained in Example 2

XRPD pattern simulation from 5 to 50° 2-Theta was performed using Mercury CCDC software:
SCAN: 5.0/50.0/0.02/0.1(sec), Cu, I(p)=10000, May 14, 2024 04:44 p
PEAK: 15(pts)/Parabolic Filter, Threshold=3.0, Cutoff=0.1%, BG=3/1.0, Peak-Top=Summit
NOTE: Intensity=Counts, 2T(0)=0.0(deg), Wavelength to Compute d-Spacing=1.54184 (Cu/K-average)

TABLE 3
simulated peak list (example 6)

| Peak | Pos. | |
|---|---|---|
| number | [°2 Θ] | Rel. Int. [%] |
| 1 | 8.217 | 10.5 |
| 2 | 9.524 | 37.5 |
| 3 | 10.178 | 14.6 |
| 4 | 11.181 | 19.6 |
| 5 | 12.421 | 2.8 |
| 6 | 13.739 | 27.2 |
| 7 | 14.563 | 45.5 |
| 8 | 15.000 | 32.4 |
| 9 | 15.698 | 4.4 |
| 10 | 16.619 | 45.9 |
| 11 | 17.162 | 2.2 |
| 12 | 17.738 | 23.5 |
| 13 | 18.783 | 83.2 |
| 14 | 18.958 | 72.5 |
| 15 | 19.681 | 10 |
| 16 | 19.84 | 15.2 |
| 17 | 20.437 | 100 |
| 18 | 20.779 | 15.6 |
| 19 | 21.602 | 21.6 |
| 20 | 21.958 | 81 |
| 21 | 22.181 | 65 |
| 22 | 22.441 | 22.2 |
| 23 | 22.662 | 37.8 |
| 24 | 22.957 | 30.1 |
| 25 | 23.562 | 34 |
| 26 | 24.079 | 15.8 |
| 27 | 24.377 | 3.8 |
| 28 | 24.804 | 5.5 |
| 29 | 25.058 | 22.2 |
| 30 | 25.28 | 55.3 |
| 31 | 26.179 | 12.3 |
| 32 | 26.539 | 6 |
| 33 | 27.003 | 32.1 |
| 34 | 27.678 | 2.7 |
| 35 | 28.803 | 42.4 |
| 36 | 29.379 | 14.7 |
| 37 | 29.757 | 4.6 |
| 38 | 30.262 | 17.5 |
| 39 | 30.658 | 18 |
| 40 | 30.957 | 8.4 |
| 41 | 31.462 | 4.1 |
| 42 | 31.761 | 14.2 |
| 43 | 32.56 | 5.4 |
| 44 | 32.842 | 3.9 |
| 45 | 33.281 | 10.3 |
| 46 | 33.718 | 3.5 |
| 47 | 33.978 | 6.2 |
| 48 | 34.703 | 18.9 |
| 49 | 35.762 | 1.8 |
| 50 | 36.339 | 11 |
| 51 | 36.962 | 4.3 |
| 52 | 37.458 | 1.1 |
| 53 | 37.784 | 7.3 |
| 54 | 38.278 | 25.1 |
| 55 | 38.401 | 25.6 |
| 56 | 39.58 | 7.9 |
| 57 | 40.059 | 8.3 |
| 58 | 40.618 | 11.1 |
| 59 | 40.962 | 6.2 |
| 60 | 41.539 | 4.8 |
| 61 | 41.919 | 5.4 |
| 62 | 42.482 | 1.2 |
| 63 | 43.12 | 4.3 |
| 64 | 44.024 | 2.2 |
| 65 | 44.3 | 5.4 |
| 66 | 44.478 | 5.2 |
| 67 | 44.74 | 4.1 |
| 68 | 45.082 | 11.6 |
| 69 | 45.562 | 3.9 |
| 70 | 46.1 | 6.3 |
| 71 | 46.321 | 3.3 |
| 72 | 47.263 | 1.2 |
| 73 | 48.482 | 2.7 |
| 74 | 48.94 | 3.9 |
| 75 | 49.297 | 3.4 |

### ABBREVIATIONS

Volume (V): Liter per kilogram of (R)-1-(3-(3-(4-aminopyrimidin-2-yl)-5-chlorophenyl)morpholino)prop-2-en-1-one

What is claimed is:

1. A pharmaceutical dosage form in the form of a tablet for oral administration comprising crystalline form I of (R)-1-(3-(3-(4-aminopyrimidin-2-yl)-5-chlorophenyl)morpholino)prop-2-en-1-one having an X-ray powder diffraction pattern comprising characteristic peaks at about 16.7, 18.9, 20.5, 22.1, and 25.4 degrees 2-theta, together with a pharmaceutical excipient.

2. The pharmaceutical dosage form according to claim 1, wherein the crystalline form I of (R)-1-(3-(3-(4-aminopyrimidin-2-yl)-5-chlorophenyl)morpholino)prop-2-en-1-one further has an X-ray powder diffraction pattern comprising characteristic peaks at about 9.6, 14.6, 16.7, 17.8, 18.9, 20.5, 22.1, 22.2, 25.4, and 28.9 degrees 2-theta.

3. A crystalline form I of (R)-1-(3-(3-(4-aminopyrimidin-2-yl)-5-chlorophenyl)morpholino)prop-2-en-1-one having an X-ray powder diffraction pattern comprising characteristic peaks at about 16.7, 18.9, 20.5, 22.1, and 25.4 degrees 2-theta.

4. The crystalline form I of (R)-1-(3-(3-(4-aminopyrimidin-2-yl)-5-chlorophenyl)morpholino)prop-2-en-1-one of claim 3, further having an X-ray powder diffraction pattern comprising characteristic peaks at about 9.6, 14.6, 16.7, 17.8, 18.9, 20.5, 22.1, 22.2, 25.4, and 28.9 degrees 2-theta.
